(19) United States
(12) Patent Application Publication
(10) Pub. No.: US 2005/0026270 A1
(43) Pub. Date: Feb. 3, 2005

(54) Stable subtilin and methods of manufacture

(75) Inventor: J. Norman Hansen, College Park, MD (US)

Correspondence Address: MILLEN, WHITE, ZELANO & BRANIGAN, P.C., 2200 CLARENDON BLVD., SUITE 1400, ARLINGTON, VA, 22201, US

(73) Assignee: University of Maryland, College Park, MD (US)

(21) Appl. No.: 10/636,586

(22) Filed: Aug. 8, 2003

Related U.S. Application Data
(63) Continuation of application No. 09/868,568, filed on Jun. 20, 2001, now abandoned.

(51) Int. Cl. C12N 9/56

(52) U.S. Cl. 435/222

(57) ABSTRACT

The present invention relates to stable subtilin and methods of producing it. A preferred method comprises one or more of the following steps: applying an acidified composition, comprising a subtilin having a naturally-occurring amino acid sequence, to a hydrophobic interaction column under conditions effective for the subtilin to bind to said column; and eluting said subtilin from the column with an effective elution reagent to form a composition comprising stable subtilin.

This application claims the benefit of U.S. Provisional No. 60/113,843, filed Dec. 24, 1998 which is hereby incorporated by reference in its entirety.

### DESCRIPTION OF THE INVENTION

The present invention relates to compositions and methods of making a stable lantibiotic, such as nisin or subtilin, preferably subtilin. Nisin is a 34-residue long peptide produced by Lactococcus lactis. Subtilin is a 32-residue long peptide produced by Bacillus subtilis. Subtilin sequences are disclosed in, e.g., U.S. Pat. Nos. 5,914,250, 5,885,811, 5,861,275, 5,576,420, 5,516,682, and 5,218,101.

The biosynthesis of these gene encoded lantibiotics involves several post-translational modifications including dehydration of serines and threonines, formation of thioether cross-linkages between dehydro residues and cysteines, translocation, removal of a leader sequence, and/or release of the mature antimicrobial peptide into the extracellular medium.

A stable subtilin in accordance with the present invention is a subtilin which possesses improved subtilin stability in comparison to a subtilin in the prior art, where the stable subtilin and the prior art subtilin have the same amino acid sequence, preferably a naturally occurring amino acid sequence as disclosed in, e.g., U.S. Pat. Nos. 5,576,420 and 5,861,275. Improved stability refers to subtilin's resistance to losing activity when subjected to certain conditions. In particular, subtilin prepared according to Jensen and Hirschman, Arch. Biochem., 4:197-309, 1994, is light-sensitive and stores poorly. For example, the subtilin of Jensen and Hirschman loses activity rapidly when stored at 4° C. or 20° C. After 5 days of storage at 4° C., a subtilin prepared according to Jensen and Hirschman shows only a minimal amount of activity, e.g., less than 5%. However, a stable subtilin in accordance with the present invention, when stored for 5 days at the same temperature retains substantially all its activity, e.g., greater than 50%, more preferably, 75% or higher, or 90% or higher. Similarly, exposure of a prior art subtilin to light results in a rapid loss of activity. The phrase. “stable subtilin” refers to this improved stability in accordance with the present invention.

Preferably, a stable subtilin can mean a subtilin which has been purified by hydrophobic interaction chromatography (HIC) and thus can also be referred to as a HIC-purified stable subtilin. The term “purified” as used herein refers to a composition that which has been subjected to fractionation, e.g., HIC-purified stable subtilin is, e.g., about 10-fold, preferably about 20-fold, more preferably about 50-fold more concentrated than prior to its application to an HIC column. A stable subtilin can comprise subtilins which have been post-translationally-modified, e.g., having a succinyl residue at its N-terminus.

The improved stability of subtilin can be measured in variety of ways, including, by looking directly at its structure, e.g., by NMR, or by assaying one or more of its biological activities. For instance, subtilin inhibits spore outgrowth and inhibits the growth of proliferating bacterial cells, especially gram-negative bacteria. These assays can be performed as described in, e.g., Liu and Hansen, Appl. Environ. Microbiol., 59:648-651, 1993; Morris et al, J. Biol. Chem., 259, 13590-13594 (1984); Appl. Environ. Microbiol, 42, 958-962 (1981). A stable subtilin possesses more biological activity than a prior art subtilin. e.g., about 5-fold, about 10-fold, about 20-fold, about 50-fold, about 60-fold, or more, when measured after storage, e.g., after storage at 4° C. or 20° C. for 2, 3, 4, 5, or 7 days, 1 week or more, 2 weeks or more, 1 month or more, etc., i.e., it has an improved storage or shelf-life.

Biological activity in inhibiting spore outgrowth can be carried out by adding heat-shocked B. cereus spores, or other target spores, to a final concentration of 75 μg/ml in 1% tryptone-100 mM Tris-phosphate buffer, pH 6.8, with a total volume of 2 ml and incubating the mixture in 15 ml. polypropylene tubes in a rotating drum shaker at 37 C. at 15 rpm for 3 hours. A series of tubes, each containing a different concentration can be assayed. After incubation, the amount of inhibition can be assessed by phase contrast microscopy, which allows spores at different states of germination and outgrowth to be distinguished. For example, in the absence of inhibitor, spores were vegetative in about an hour. Spores were judged to be inhibited if more than 50% were still in early outgrowth stage after the three hour incubation period.

Biological activity against the growth of vegetative cells by measuring the growth of cells in the presence and absence of subtilin. For example, vegetative cells can be obtained by incubating heat-shocked B. cereus T spores under the aforementioned conditions in the absence of inhibitor for about 2 hours at 37° C., when the spores were in the vegetative state. Various amounts of the antimicrobial peptides were added to tubes containing the vegetative cell culture and incubated for one additional hour. The turbidity of the cell suspension was measured (in Klett units) before and after the final incubation by using a Klett-Summerson calorimeter with a green (500- to 570-nm-range) filter, and the integrity of the cells was determined by phase-contrast microscopy.

In a preferred embodiment, the present invention relates to methods of isolating a composition comprising a stable subtilin, comprising one or more of the following steps: culturing a Bacillus subtilis under effective conditions to express subtilin; applying a composition, comprising a subtilin having a naturally-occurring amino acid sequence, to a hydrophobic interaction column under conditions effective for the subtilin to bind to said column; eluting said subtilin from the column with an effective elution reagent to form a composition comprising stable subtilin; performing reverse phase HPLC on said stable subtilin.

In performing a method of the present invention, a starting composition comprising a subtilin can be obtained from any suitable source. In preferred embodiments, a starting composition is obtained by culturing a cell comprising a subtilin gene under conditions effective for expression of subtilin. By the term, “expression,” it is meant that the cell carries out the cellular processes which result in the production of a subtilin polypeptide. Effective conditions for expression include any culture conditions which are suitable for achieving production of the subtilin polypeptide by the cell, including effective temperatures, pH, medias, cell densities, culture dishes, flasks, or other receptacles for growing cells, additives (e.g., cycloheximide, protease inhibitors, etc.).

Any cell comprising a subtilin gene can be used in accordance with the present invention, including naturally-occurring and genetically-engineered cells. A preferred cell which expresses subtilin is Bacillus subtilis ATCC No. 6633, or derivatives thereof. The present invention also relates to the production of subtilin by organisms which have been genetically-engineered to express subtilin. In a preferred embodiment, B. subtilis strain 168, or substrain B. subtilis strain BR151 (ATCC No. 33677) is transformed with an expressible subtilin gene, e.g., as described in Liu and Hansen, J. Bacteriol., 173:7387-7390, 1991, to form strain LH45 which was deposited under the terms of the Budapest Treaty and assigned ATCC No. 202127. All restrictions imposed by the depositor on the availability to the public of the deposited material will be irrevocably removed upon granting of a patent. By the term “expressible gene,” it is meant that the gene is effective for production the subtilin polypeptide, i.e., transcription and translation of the subtilin gene. For example, the subtilin coding sequence is operably linked to an expression control. The phrase “expression control sequence” means a nucleic acid sequence which regulates expression of a polypeptide coded for by a nucleic acid to which it is operably linked. Expression can be regulated at the level of the MRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can be heterologous or endogenous to the normal gene.

Any suitable growth media can be used to culture the subtilin-expressing cells, e.g., media comprising, nitrogen sources, such as yeast extracts, soy bean tripticase, peptone, salts, metal ions, citric acid, buffers, carbohydrates, such as glucose, glycerol, lactose, sucrose, molasses, chalk, phosphates, ammonium sulfate, oil. See, examples for specific embodiments.

A composition comprising a subtilin can be pre-treated prior to its application to a hydrophobic interaction column in any desired manner. For example, the composition can be diluted, concentrated, precipitated, filtered, filtered through a porous membrane to separate insoluble debris, acidified, centrifuged, heat-treated, etc. It can be supplemented with additives, such as protease inhibitors, anti-oxidants, detergents, salts, chaotropic agents, chelating agents such as EDTA or EGTA, metal ions, cofactors, etc. In the example below, prior to applying it the column, the culture media containing subtilin is acidified to pH 2.5 with 85% phosphoric acid (14.6 M) to form an acidified composition. Acidification can be performed with any suitable acid, e.g., acetic acid, carboxylic acids, glycine HCl, etc.

In preferred embodiments of the invention, the subtilin is secreted into the culture medium. The cells can be separated from the culture medium by low-speed centrifugation. The resulting supernatant comprising the subtilin can then be harvested for further manipulations. Centrifugation to remove cells can be performed prior to acidification or after it. Alternatively, the cells can be homogenized to form a homogeneously composition comprising subtilin.

In preferred embodiments of the invention, a stable subtilin is obtained by reversed phase chromatography, preferably hydrophobic interaction chromatography (HIC) Hydrophobic interaction chromatography uses a nonpolar stationary phase and a polar mobile phase to separate compounds according to their hydrophobicity, using a chromatographic support material having a hydrophobic surface. The binding of a polypeptide to a hydrophobic interaction column can be induced by the addition of high salt concentrations to the sample comprising the polypeptide. The polypeptides in the sample effectively “salt out” on to the hydrophobic surface of the chromatography support. Binding strength can be modulated by manipulating the salt concentration and choosing supports with different hydrophobicities. The binding interaction between the hydrophobic column surface and the polypeptide is reversible. Reducing the salt concentration, changing ionic strength, or adding agents which disrupt the hydrophobic binding can be used to elute the polypeptide from the hydrophobic support surface.

Any suitable chromatographic material can be used. For example, a variety of different chromatographic materials supports are commercially available which have hydrophobic ligands attached to a chromatographic support, e.g., having an alkyl chain ranging from about two to twenty-four or more carbons in length, linear or branched, and can contain or terminate in other hydrophobic groups such as a phenyl ring. Increasing chain length results in media with greater hydrophobic character. Commonly used ligands are phenyl-, butyl-, and octyl- residues. Commercially available hydrophobic interaction chromatographic materials includes, e.g., POROS HP2 (surface covered with phenyl groups), POROS PE (surface coated with phenyl ether groups), POROS ET (surface coated with ether groups), Bio-Rad Macro-Prep HIC Supports, Bio-Rad Methyl HIC support, Bio-Rad-t-butyl HIC support, Bio-rad Econo column butyl-650 m, TosoHaas TSK-GEL® HIC Columns, TosoHaas TOKYOPEARL® HIC, Fractogel® EMD Propyl (S), Fractogel® EMD Phenyl I (S), IEC PH-814, PolyMETHYL A™, PolyETHYL A™, PolyPROPYL A™, PolyBUTYL A™, HiPrep™ 16/10 Phenyl, HiPrep™ 16/1-Butyl, HiPrep™ 16/10 Octyl, etc. See, also, U.S. Pat. No. 5,641,403. In addition to resins that react strictly through hydrophobicity, there are materials that work via a combination of hydrophobic and ionic interactions such as amino-hexyl Sepharose (LKB/Pharmacia, Piscataway, N.J.) which incorporates an amino group at the end of a six carbon alkyl chain as the active function.

As mentioned, binding of polypeptides to the hydrophobic column can be induced by high salt concentrations. Thus, in preferred embodiments of the invention, high salt is added to a composition comprising subtilin in order to increase its salt concentration and facitiate the subtilin's binding to the HIC column. Any suitable salt can be used, including, e.g, NaCl, such as 0.75 M, 1 M, 1.2 M, 1.5 M, 1.75 M, 2 M, 4 M, etc., ammonium sulfate, such as 0.75 M, 1 M, 1.2 M, 1.5 M, 1.75 M, 2 M, 4 M, etc., sodium acetate, sodium phosphate, etc.

In practice, the strength of the hydrophobic interactions is also influenced by the ionic strength, pH and polarity of the solvent. For example, a high concentration (i.e., 4 molar) of ammonium sulphate in the solvent would promote hydrophobic interaction and, hence, binding, between the resin and the hydrophobic domains of the solute proteins.

Once the subtilin is bound or adsorbed to the HIC column, and the other material in the composition which do not bind to the column are washed out, the subtilin can be removed from the column by interfering its interactions with the hydrophobic ligands present on the column support. The subtilin can thus be eluted from the column with an effective elution reagent. By “effective elution reagent,” it is meant any agent which can be used to remove the subtilin from the column, including, compounds, compositions, mixtures, etc. Generally, any effective reagent and/or procedure can be used to elute a subtilin from the resin, including, but not limited to, a buffer with lower ionic strength, chaotropic ions, polarity-lowering additives, such as ethylene glycol, detergents, methanol, acetonitrile, THF, and/or dichlormethane. In preferred embodiments, the elution reagent is acetonitrile, e.g., 30% acetonitrile.

The stable subtilin eluted from the HIC column can be further purified according to any suitable methods, including, chromatography, affinity chromatography, thin-layer chromatography, HPLC, SDS-PAGE electrophoresis. For a discussion of these methods, e.g., Principles and Practice of Modern Chromatographic Methods, Robards et al., Academic Press, 1994; Gas and Liquid Chromatography in Analytical Chemistry, R. M. Smith, Wiley and Sons, 1988; Journal of Chromatography Library, e.g., Volume 27, Instrumental Liquid Chromatography, N. A. Parris, Elsevier, 1984. See, also examples.

A stable subtilin can also be isolated according to prior art procedures in combination with hydrophobic interaction chromatography, e.g. by a modification of the previously published procedures, Jensen and Hirschman, Arch. Biochem., 4, 197-309 (1944). In this procedure, cells are cultured in a medium A (Bannerjee and Hansen, J Biol Chem., 263, 9508-9514, 1988) containing 10% sucrose and incubated with good aeration for 30-35 hours at 35° C. The culture is then acidified to pH 2.8 with phosphoric acid and heated in an autoclave at 121° C. for 3 min to inactivate proteases, and cooled to room temperature. One-half volume of n-butanol is added, stirred at 4° C., for 2 hours, and centrifuged. 2.5 volume of acetone is added to the supernatant, allowed to stand at −20° C. for at least 2 hours, and then centrifuged. Most of the pellet is subtilin which is washed with 95% ethanol, briefly lyophilized, and dissolved in 0.1% trifluoroacetic acid. This is immediately purified by RP-HPLC as described previously for nisin (Liu, W. and J. N. Hansen, “Some Chemical and Physical Properties of Nisin, a Small Protein Antibiotic Produced by Lactococcus lactis”. Appl. Environ. Microbiol. (1990) 56:2551-2558.), which employs a trifluoroacetic acid-water-acetonitrile gradient. Subtilin elutes slightly later than nisin in this gradient. Peaks are collected, lyophilized and stored at −80° C.

A subtilin in accordance with the present invention has bactericidal and bacteriostatic activity, e.g., preventing spore outgrowth and inhibiting bacterial cell growth proliferation, especially gram-negative bacteria. As a result, subtilin is useful in treating microbial infections, as a food preservative, and in other applications where antibiotic activity is useful.

To treat microbial infections, especially those caused by gram-native bacteria, subtilin can be used analogously to other antimicrobial drugs, especially antibiotics, as described in, e.g., Reminigton's Pharmaceutical Sciences, Eighteenth Edition, 1990, Chapter 62. Any effective route of administration can be used, e.g., topical or oral.

Subtilin can be used alone, or in combination with other agents, such as other bactericides, in combination with nisin or any effective antibiotic. A stable subtilin can also be used as a food preservative to keep food safe for consumption, by inhibiting or prevents nutrient deterioration and organoleptic changes produced by bacterial activity. The subtilin can be directly incorporated into the food, such as a wine, fish, cheese, dairy product, or processed meat, or it can be used in food packaging materials. See, e.g., U.S. Pat. Nos. 4,584,199, 4,597,972, 4,980,163, and 5,573,800. Subtilin can also be used in mouthwash. A composition comprising a stable subtilin can contain buffers, carriers, etc.

The present invention relates to methods of treating a bacterial infection in a host, comprising administering an effective amount of a stable subtilin to a host in need thereof, and methods of preserving food, comprising adding an effective amount of a stable subtilin to a food in need thereof. By the phrase, “effective amount,” it is meant any amount of subtilin which is useful in achieving the claimed purpose, e.g., treating an infection or preserving food. The effective amounts can be determined routinely, and/or in analogy to nisin. Prior art subtilin, e.g., having a naturally-occurring sequence, preferably not modified by genetic engineering, was so unstable that it was not useful. The improved stability of stable subtilin makes it useful in the aforementioned methods.

### EXAMPLES

Subtilin was purified as follows: One liter of medium A containing 2% sucrose was inoculated with 5-10 ml of overnight culture grown in PAB for 24-28 hr at 37° C. with vigorous shaking. The subtilin production was monitored by the pink-brown color, fruity odor, and pH (5.9-6.9) of the medium. All of the purification steps were performed at room temperature (RT). The culture was acidified to pH 2.5 with 85% phosphoric acid(14.6 M), centrifuged at 10,000×g for 20 min to remove cells, and the subtilin in supernatant fraction purified to homogeneity in two steps. First, the culture supernatant with salt concentration adjusted to 1 M NaCl, was loaded onto a butyl-650 m hydrophobic interaction column (Bio-Rad Econo-column, V=26 ml) equilibrated with 50 mM of sodium acetate, pH 4.0 containing 1 M NaCl. Proteins not bound to the column were washed out extensively with the loading buffer, and then the bound proteins, including the subtilin, were eluted with 30% acetonitrile. This was lyophilized, redissolved in water with 0.1% trifluoroacetic acid, and further purified by reverse-phase HPLC. This was done in two-step linear gradient, with the first step going from 0 to 25% acetonitrile over 30 minutes, with the second step going from 25-35% acetonitrile over 30 minutes. All flow rates were 1.2 ml/min, and all the solvents contained 0.1% TFA in addition to acetonitrile. The active fractions were pooled, rechromatographed and the single peak with antibiotic activity collected and lyophilized before further analysis. The absorbance was monitored at 254 nm (for the dehyro amino acids) and 280 nm (for the aromatic amino acids) or 214 nm (for peptide bonds).

Medium A was prepared by combining 780 ml of solution I (20 g of sucrose in 780 ml of distilled water), 200 ml of solution II (53.5 g of citric acid, 20.0 g of Na₂SO₄, 25.0 g of yeast extract, and 21.0 g of (NH₄)₂HPO₄ in 11 of distilled water, with the pH adjusted to 6.9 with 14.8 N NH₄OH), 10 ml of solution III (7.62 g KCl, 41.8 g MgCl₂.6H2O, 0.11 g ZnCl₂, 0.18 g MnCl₂.4H₂O dissolved in 100 ml of distilled water), and 10 ml of solution IV (0.25 g of FeCl₃ dissolved in 100 ml of distilled water). The solutions I, II and II were autoclaved separately and combined after cooling to RT to avoid the precipitation of FeCl₃ as Fe(OH)₃. The solution IV was sterilized by filtering through nitrocellulose filter disk (0.22 mm).]

The obtained subtilin lantibiotic can be used alone as an antimicrobial compound or in combination with other lantibiotics and antibiotics for treating or preventing microbial growth.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents, publications, nucleic acids having sequence identification numbers, etc., cited above and in the figures are hereby incorporated in their entirety by reference.

From the foregoing description, one skilled in the art can easily ascertain the essential characteristics of this invention, and without departing from the spirit and scope thereof, can make various changes and modifications of the invention to adapt it to various usages and conditions.

1. A method for isolating a composition comprising a stable subtilin,
applying an acidified composition, comprising a subtilin having a naturally-occurring amino acid sequence, to a hydrophobic interaction column under conditions effective for the subtilin to bind to said column; and
eluting said subtilin from the column with an effective elution reagent to form a composition comprising stable subtilin.

2. A method of claim 1, further comprising culturing a Bacillus subtilis under effective conditions for expression of subtilin.

3. A method of claim 2, wherein said Bacillus is strain BR151 which comprises an expressible subtilin gene.

4. A method of claim 3, wherein said Bacillus strain is LH45.

5. A method ofclaim 1, further comprising acidifying a culture medium comprising a subtilin prior to said applying.

6. A method of claim 1, wherein said column is a butyl 650 m hydrophobic interaction column and the elution reagent is acetonitrile.

7. A method of claim 1, further comprising performing reverse phase HPLC on said stable subtilin.

8. A method of claim 1, wherein said stable subtilin retains 90% of its biological activity after storage at 4° C. for 5 days.

9. A composition comprising stable subtilin and elution reagent produced by a method comprising:
applying an acidified composition, comprising a subtilin having a naturally-occurring amino acid sequence, to a hydrophobic interaction column to bind said subtilin to said column; and eluting said subtilin from the column with an effective elution reagent.

10. A composition as in claim 9 wherein the acidified composition comprising a stable subtilin having a naturally-occurring amino acid sequence is derived from culturing a Bacillus subtilis to express subtilin.

11. A composition as in claim 10 wherein said Bacillus is strain BR151 which comprises an expressible subtilin gene.

12. A composition as in claim 10 wherein said Bacillus is LH45.

13. A combination of stable subtilin having a naturally-occurring amino acid sequence and another bactericide.

14. Subtilin having a naturally-occurring amino acid sequence which retains 90% or more of its biological activity after storage at −4° C. for five days or more.

15. Cancelled

16. A method of treating a bacterial infection in a host, comprising administering an effective amount of a stable subtilin to a host in need thereof.

17. A method of preserving food, comprising adding an effective amount of a stable subtilin to a food in need thereof.

18. A composition comprising stable subtilin having a naturally-occurring amino acid sequence produced by a method comprising:
applying an acidified composition, comprising a subtilin having a naturally-occurring amino acid sequence, to a hydrophobic interaction column to bind said subtilin to said column; and
eluting said subtilin from the column with an elution reagent to form a composition comprising stable subtilin having a naturally-occurring amino acid sequence.

19. A composition as in claim 18 wherein the acidified composition comprising a subtilin having a naturally-occurring amino acid sequence is derived from culturing a Bacillus subtilis to express subtilin.

20. A composition as in claim 19 wherein said Bacillus is strain BR151 which comprises an expressible subtilin gene.

21. A composition as in claim 19 wherein said Bacillus is strain is LH45.

22. A composition comprising stable subtilin having a naturally-occurring amino acid sequence produced by
culturing a Bacillus subtilis, with an expressible subtilin gene, selected from the group consisting of strain BR151 and LH45 to express subtilin having a naturally-occurring amino acid sequence;
acidifying said culture of Bacillus subtilis with stable subtilin having a naturally-occurring amino acid sequence;
applying said acidified culture medium of Bacillus subtilis, comprising a subtilin having a naturally-occuring amino acid sequence, to a butyl 650 m hydrophobic interaction column to bind said subtilin to said column; and
eluting said subtilin from the column with acentonitrile to form a composition comprising stable subtilin having a naturally-occurring amino acid-sequence.

23. A compositin as in claim 18 wherein said method further comprises performing revers phase HPLC of the eluted stable subtilin having a naturally-occurring amino acid-sequence.

24. A composition as in claim 22 wherein said method further comprises performing reverse phase HPLC of the eluted stable subtilin having a naturally-occurring amino acid sequence.
